(19) United States
(12) Patent Application Publication
(10) Pub. No.: US 2010/0305444 A1
(43) Pub. Date: Dec. 2, 2010

(54) ULTRASONIC DIAGNOSIS APPARATUS, IMAGE DISPLAY APPARATUS, IMAGE DISPLAY METHOD, AND DISPLAY METHOD

(76) Inventors: Tomokazu FUJII, Nasushiobara-shi (JP); Yoshihisa Yoshioka, Nasushiobara-shi (JP); Masato Oonuki, Yaita-shi (JP)

Correspondence Address: OBLON, SPIVAK, MCCLELLAND MAIER & NEUSTADT, L.L.P., 1940 DUKE STREET, ALEXANDRIA, VA, 22314, US

(21) Appl. No.: 12/790,364

(22) Filed: May 28, 2010

(30) Foreign Application Priority Data
May 29, 2009 (JP) 2009-131192

(51) Int. Cl. A61B 8/14

(52) U.S. Cl. 600/443

(57) ABSTRACT

According to one embodiment, an ultrasonic diagnosis apparatus includes an ultrasonic probe, a generating unit, a first housing, a second housing, a display unit, a recognition unit, and a display control unit. The generating unit generates ultrasonic image data. The first housing includes an operation panel for issuing an operation instruction. The second housing is connected to the first housing and includes a screen. The display unit displays a display image on the screen. The display image includes at least one of an image display area for the ultrasonic image and a panel display area for a display panel. The recognition unit recognizes a connection state between the first housing and the second housing. The display control unit changes at least one of display positions, sizes, and shapes of the image display area and the panel display area on the display image in accordance with the recognized connection state.

### CROSS-REFERENCE TO RELATED APPLICATIONS

This application is based upon and claims the benefit of priority from Japanese Patent Application No. 2009-131192, filed May 29, 2009; the entire contents of which are incorporated herein by reference.

### FIELD

Embodiments described herein relate generally to a compact ultrasonic diagnosis apparatus having a notebook PC (notebook personal computer) structure, an image display apparatus, image display method, and display method.

### BACKGROUND

Ultrasonic diagnosis apparatuses are widely used for examinations in examination rooms and hospital wards owing to their characteristics such as non-invasiveness and portability. With the recent advances in liquid crystal techniques, HDDs (Hard Disk Drives), and batteries used for notebook PCs, such techniques have been applied to ultrasonic diagnosis apparatuses. With this trend, compact, lightweight ultrasonic diagnosis apparatuses having a notebook PC structure have been under development. In addition, such compact, lightweight ultrasonic diagnosis apparatuses have been used in an environment in which they are moved to various places such as an examination room, a hospital ward, an operating room, and an outdoor place for diagnosis.

Such a notebook PC type ultrasonic diagnosis apparatus includes a display housing including a screen and an operation housing including an operation panel. With a reduction in the size of an ultrasonic diagnosis apparatus, the size of the screen decreases. A display image displayed on the screen includes an image display area for an ultrasonic image and a panel display area for a display panel. Various types of switches on the panel display area each are inevitably equal in size to that in a large-size apparatus regardless of the size of the apparatus. Therefore, the ultrasonic image on the image display area is reduced depending on the size of the screen. This is a problem in diagnosis.

With the miniaturization of an ultrasonic diagnosis apparatus, the size of the operation panel decreases. For this reason, an operation panel is reduced in size or functions are integrated to make the panel have the same functions as those of a large-size apparatus. For example, the size of each switch is reduced, switchable functions are assigned to one switch, or a smaller number of switches are displayed on the panel display area.

In general, a large-size ultrasonic diagnosis apparatus allows to independently move the screen and the operation panel. Therefore, it is possible to smoothly execute ultrasonic examination by placing the screen at a position where persons (an observer and an operator) can see it and always placing the operation panel at a position near the operator. In the case of a compact ultrasonic diagnosis apparatus, the screen and the operation panel are fixed at nearby positions. If, therefore, the screen is placed at a position where persons can see it, the operation panel is inevitably placed at a position far from the operator. When the screen is equipped with a touch panel, in particular, the touch panel is physically far from the operator or observer. When the touch panel is far from the operator, it is difficult to operate the ultrasonic diagnosis apparatus. When the touch panel is far from the observer, it is difficult to observe an ultrasonic image. Therefore, the throughput of ultrasonic examination using a compact ultrasonic diagnosis apparatus is lower than that using a large-size ultrasonic diagnosis apparatus.

### BRIEF DESCRIPTION OF THE DRAWINGS

FIG. 1 is a perspective view of an ultrasonic diagnosis apparatus according to an embodiment;

FIG. 2 is a perspective view of the tablet mode of the ultrasonic diagnosis apparatus in FIG. 1;

FIG. 3 is a functional block diagram of the ultrasonic diagnosis apparatus according to the first example in FIG. 1;

FIG. 4 is a view showing an example of the layout of a display image in an open mode, which is displayed by a display unit in FIG. 3;

FIG. 5 is a view showing an example of the layout of a display image in a tablet mode, which is displayed by the display unit in FIG. 3;

FIG. 6 is a view showing an example of the layout of a display image in the tablet mode, which is displayed by the display unit in FIG. 3;

FIG. 7 is a view showing an example of the layout of a display image in the tablet mode, which is displayed by the display unit in FIG. 3;

FIG. 8 is a perspective view showing how only an operator is using the ultrasonic diagnosis apparatus in FIG. 3 in the open mode;

FIG. 9 is a perspective view showing how an operator and an observer are using the ultrasonic diagnosis apparatus in FIG. 3 in the open mode;

FIG. 10 is a perspective view showing how an operator and an observer are using the ultrasonic diagnosis apparatus in FIG. 3 in the tablet mode, and only an image display area is displayed;

FIG. 11 is a perspective view showing how an operator and an observer are using the ultrasonic diagnosis apparatus in FIG. 3 in the tablet mode, and an image display area and a reduced panel display area are displayed;

FIG. 12 is a perspective view showing how an operator and an observer are using the ultrasonic diagnosis apparatus in FIG. 3 in the tablet mode, and an image display area and two reduced panel display areas are displayed;

FIG. 13 is a functional block diagram of an ultrasonic diagnosis apparatus according to the second example in FIG. 1;

FIG. 14 is a conceptual view showing how a mouse is connected to the ultrasonic diagnosis apparatus in FIG. 1;

FIG. 15 is a view showing a standard trackball;

FIG. 16 is a view showing an example of the examination sequence layout on the panel display area, which is displayed by a display unit in FIG. 11 in the open mode;

FIG. 17 is a view showing an example of the examination sequence layout on the panel display area, which is displayed by the display unit in FIG. 11 in the tablet mode while the mouse is connected;

FIG. 18 is a view showing an example of the examination sequence layout on the panel display area, which is displayed by the display unit in FIG. 11 in the tablet mode while the mouse is disconnected; and

FIG. 19 is a conceptual view showing a state in which a wireless mouse is connected to the ultrasonic diagnosis apparatus in FIG. 1.

### DETAILED DESCRIPTION

In general, according to one embodiment, an ultrasonic diagnosis apparatus according to this embodiment includes an ultrasonic probe, a generating unit, a first housing, a second housing, a display unit, a recognition unit, and a display control unit. The ultrasonic probe configured to transmit and receive an ultrasonic wave. The generating unit configured to generate ultrasonic image data based on an echo signal from the ultrasonic probe. The first housing connected to the ultrasonic probe and including an operation panel for issuing an operation instruction associated with ultrasonic examination. The second housing connected to the first housing and including a screen. The display unit configured to display a display image on the screen, the display image including at least one of an image display area for the generated ultrasonic image and a panel display area for a display panel. The recognition unit configured to recognize a connection state between the first housing and the second housing. The display control unit configured to change at least one of display positions, sizes, and shapes of the image display area and the panel display area on the display image in accordance with the recognized connection state.

The ultrasonic diagnosis apparatus according to this embodiment will be described below with reference to the views of the accompanying drawing.

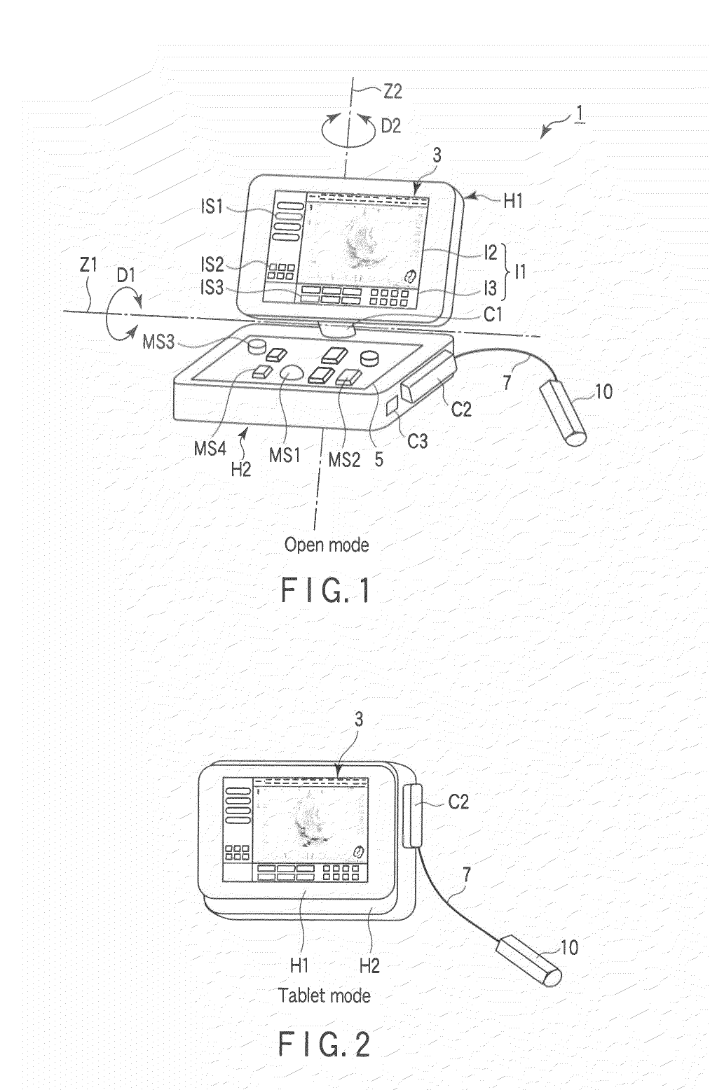

FIG. 1 is a perspective view of an ultrasonic diagnosis apparatus 1 according to this embodiment. As shown in FIG. 1, the ultrasonic diagnosis apparatus 1 is equipped with a display housing H1 including a screen 3 on the upper surface, an operation housing H2 including an operation panel 5 on the upper surface, and an ultrasonic probe 10 connected to the operation housing H2 via a cable 7. The display housing H1 is connected to the operation housing H2 through a housing connection portion C1. As described above, the ultrasonic diagnosis apparatus 1 according to this embodiment is a compact, lightweight notebook PC type ultrasonic diagnosis apparatus having the screen 3 and the operation panel 5 integrally formed.

The operation housing H2 accommodates a substrate. The substrate is mounted on electronic parts to execute the functions of the ultrasonic diagnosis apparatus 1 (to be described later). The operation housing H2 includes a probe connection portion C2 for connecting the ultrasonic probe 10 to the operation housing H2. Input devices such as a mouse and a keyboard (not shown) may be connected to the operation housing H2. The operation housing H2 includes a device connection portion C3 to connect the input device to the operation housing H2.

A display unit 22 (to be described later) displays a display image I1 on the screen 3. The display image I1 includes an image display area I2 for an ultrasonic image and a panel display area I3 for a display panel. The windows associated with various kinds of applications for ultrasonic diagnosis are displayed on the image display area I2. Each window typically displays an ultrasonic image. An application window displays parameters for the application and additional data such as measurement values. A display switch group IS (display panel) for issuing various operation instructions associated with ultrasonic examination to a system control unit 30 (to be described later) is displayed on the panel display area I3. A display panel IS displays switches IS1, IS2, and IS3. The switches IS are displayed by a GUI (Graphical User Interface) technique.

The operation panel 5 includes a mechanical switch group MS for issuing various operation instructions associated with ultrasonic examination to the system control unit 30 (to be described later). More specifically, the operation panel 5 includes the switch group MS including a trackball MSI, a button MS2, a knob MS3, and a switch MS4.

With a reduction in the size of the ultrasonic diagnosis apparatus 1, the area of the operation panel 5 tends to decrease. It is therefore preferable to mount only basic switches for ultrasonic diagnosis on the operation panel 5 without any needless switches. Switches which are not mounted on the operation panel 5 may be displayed as GUI elements on the panel display area I3 of the screen 3.

The housing connection portion C1 mechanically connects the display housing H1 to the operation housing H2 so as to allow the display housing H1 and the operation housing H2 to open/close along a direction D1. The direction D1 is defined as a direction in which the display housing H1 and the operation housing H2 open and close about an opening/closing axis Z1. The opening/closing axis Z1 is parallel to the long axis of the display housing H1 and intersects with the housing connection portion C1. The housing connection portion C1 also mechanically connects the display housing H1 to the operation housing H2 so as to allow the display housing H1 and the operation housing H2 to rotate along a direction D2 around the housing connection portion C1. The direction D2 is defined as a direction around a rotating axis Z2. The rotating axis Z2 is parallel to the short axis of the display housing H1 and intersects with the housing connection portion C1. The housing connection portion C1 is implemented by, for example, a hinge. Note that FIG. 1 shows the single housing connection portion C1 which allows the housings to open/close in the direction D1 and rotate in the direction D2. However, this embodiment is not limited to this. For example, this apparatus may separately include a housing connection portion (not shown) for implementing opening/closing in the direction D1 and a housing connection portion (not shown) for implementing rotation in the direction D2.

The ultrasonic diagnosis apparatus 1 implements two different display forms based on opening/closing in the direction D1 and rotation in the direction D2 in accordance with the connection state between the screen 3 and the operation panel 5. The first display form is an open mode like that shown in FIG. 1. In the open mode, the display housing H1 and the operation housing H2 are supported to make the front of the screen 3 face the front of the operation panel 5. This open mode is a standard display form used for ultrasonic examination. The second display form is a tablet mode like that shown in FIG. 2. In the tablet mode, the display housing H1 and the operation housing H2 are folded so as to allow the screen 3 to be seen from outside the ultrasonic diagnosis apparatus 1. In other words, in the tablet mode, the display housing H1 and the operation housing H2 are folded so as to allow the back of the screen 3 face the front of the operation panel 5. To shift from the open mode to the tablet mode, the user rotates the display housing H1 in the open mode through 180° along the direction D2 and close the display housing H1 along the direction D1 so as to bring the display housing H1 into tight contact with the operation housing H2. To shift from the tablet mode to the open mode, the user performs the operation reverse to that described above. That is, the user opens the display housing H1 in the tablet mode along the direction D1 and rotates the display housing H1 through 180° along the direction D2.

The functions of the ultrasonic diagnosis apparatus 1 which are implemented by the electronic parts housed in the operation housing H2 will be separately described in the first example and the second example.

### First Example

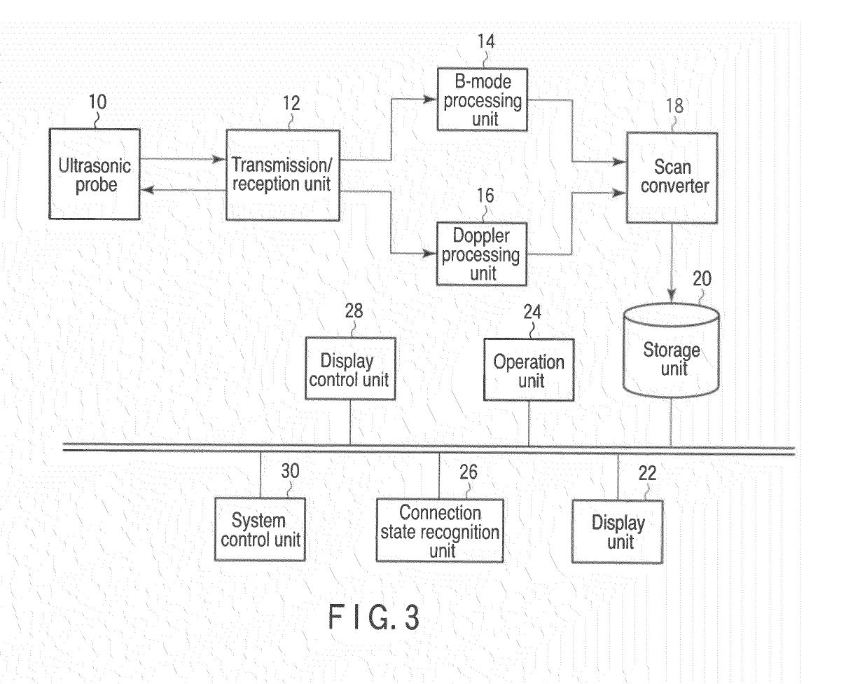

FIG. 3 is a functional block diagram of an ultrasonic diagnosis apparatus 1 according to the first example. As shown in FIG. 3, the ultrasonic diagnosis apparatus 1 includes an ultrasonic probe 10, a transmission/reception unit 12, a B-mode processing unit 14, a Doppler processing unit 16, a scan converter 18, a storage unit 20, a display unit 22, an operation unit 24, a connection state recognition unit 26, a display control unit 28, and a system control unit 30.

The ultrasonic probe 10 transmits ultrasonic waves to an subject in accordance with driving pulses from the transmission/reception unit 12. The transmitted ultrasonic waves are sequentially reflected by a discontinuity surface of the acoustic impedance of tissue in a subject. The ultrasonic probe 10 receives the reflected ultrasonic waves as an echo signal. The received echo signal is supplied to the transmission/reception unit 12 via the ultrasonic probe 10.

The transmission/reception unit 12 transmits ultrasonic waves to the subject via the ultrasonic probe 10, and receives, as an echo signal, ultrasonic waves reflected by the subject.

More specifically, the transmission/reception unit 12 includes a rate pulse generating circuit, transmission delay circuit, and driving pulse generating circuit (none of which are shown) for ultrasonic transmission. The rate pulse generating circuit repeatedly generates rate pulses for each channel at a predetermined rate frequency fr Hz (period: 1/fr sec). The delay circuit assigns each rate pulse a delay time necessary for focusing an ultrasonic wave into the form of a beam for each channel and determining transmission directivity. The pulse generating circuit applies a driving pulse to the ultrasonic probe 10 at the timing based on each delayed rate pulse.

The transmission/reception unit 12 includes an amplifier circuit, A/D converter, reception delay circuit, and adder (none of which are shown) for ultrasonic reception. The amplifier circuit receives an echo signal from the ultrasonic probe 10 and amplifies the received echo signal for each channel. The A/D converter converts the amplified echo signal for each channel from an analog signal to a digital signal. The reception delay circuit assigns the echo signal converted into the digital signal, for each channel, a delay time necessary for focusing the digital signal into the form of a beam and determining reception directivity. The adder then adds the respective echo signals assigned with the delay times. With this addition, a reflection component from a direction corresponding to the reception directivity of the echo signal is enhanced to form an ultrasonic beam in accordance with reception directivity and transmission directivity. One ultrasonic beam corresponds to one ultrasonic scanning line. The echo signals are supplied to the B-mode processing unit 14 and the Doppler processing unit 16 for each scanning line.

The B-mode processing unit 14 receives an echo signal from the transmission/reception unit 12, and detects the envelope of the logarithmically amplified echo signal to generate B-mode signal data representing the intensity of the echo signal as a luminance. The generated B-mode signal data is supplied to the scan converter 18.

The Doppler processing unit 16 frequency-analyzes an echo signal from the transmission/reception unit 12, extracts a blood flow or tissue owing to a Doppler effect and a contrast medium echo component, and generates Doppler signal data expressing the intensity of blood information such as a mean velocity, variance, power, or the like in color. The generated Doppler signal data is supplied to the scan converter 18.

The scan converter 18 generates ultrasonic image data associated with the subject based on the B-mode signal from the B-mode processing unit 14 and the Doppler signal from the Doppler processing unit 16. That is, the scan converter 18 functions as a unit to generate ultrasonic image data. More specifically, the scan converter 18 interpolates data between scanning lines by arranging the data in the memory in accordance with the position information of the B-mode signal or Doppler signal. This arrangement processing and interpolation processing will generate ultrasonic image data.

The storage unit 20 stores the ultrasonic image data generated by the scan converter 18. The storage unit 20 also stores a program for display image switching processing unique to the first example.

The display unit 22 displays a display image I1 on the screen 3. The display image I1 described above includes an image display area I2 for an ultrasonic image and a panel display area I3 for a display panel.

The operation unit 24 includes the operation panel 5. As described above, the operation panel 5 includes a mechanical switch group MS for issuing various operation instructions associated with ultrasonic examination to the system control unit 30. The operation unit 24 includes input devices such as a mouse. The operation unit 24 detects the coordinates of the cursor displayed on the display unit 22 and outputs the detected coordinates to the system control unit 30. The operation panel 5 includes a touch panel provided to cover the screen 3. The operation panel 5 detects touched and indicated coordinates by a coordinate reading principle such as an electromagnetic induction system, a magnetostriction system, or a pressure sensitive system, and outputs the detected coordinates as a position signal to the system control unit 30.

The connection state recognition unit 26 recognizes the mechanical connection state between the screen 3, i.e., a display housing H1, and the operation panel 5, i.e., an operation housing H2. Typically, the connection state recognition unit 26 recognizes electrically, magnetically, or optically, as a connection state, whether the display housing H1 and the operation housing H2 are in the open mode or the tablet mode. For example, the connection state recognition unit 26 is implemented by an optical sensor embedded in the housing connection portion (hinge) C1. The connection state recognition unit 26 may also be implemented by a combination of a magnet and a magnetic sensor. For example, the magnet is embedded in a part near the center of the upper part of the display housing H1. In this case, the magnetic sensor is embedded in a part of the operation housing H2 so as to face the magnet in the tablet mode. The magnetic sensor detects a magnetic field equal to or more than a threshold. For example, the threshold is set between a magnetic field originating from the magnet which is detected in the tablet mode and a magnetic field originating from the magnet in the open mode. The magnetic sensor detects a magnetic field generated by the magnet. It is possible to recognize whether the magnet is located near the magnetic sensor, i.e., the tablet mode is set.

The display control unit 28 changes at least one of the display positions, sizes, and shapes of the image display area I2 and panel display area I3 on the display image I1 in accordance with the mechanical connection state recognized by the connection state recognition unit 26. More specifically, upon recognizing the open mode, the display control unit 28 displays the image display area I2 and the panel display area I3 on the display image I1 at the normal display positions and with the normal sizes and shapes. Upon recognizing the tablet mode, the display control unit 28 displays, for example, only the image display area I2 on the entire screen 3.

The system control unit 30 controls all processes in the ultrasonic diagnosis apparatus 1 according to this example. The system control unit 30 reads out a dedicated program from the storage unit 20 and unarchives the program in the memory to control the respective units of the ultrasonic diagnosis apparatus 1, thereby executing display image switching processing.

Display image switching processing according to the first example which is performed under the control of the system control unit 30 will be described next in detail. Typically, the display control unit 28 holds a table which associates the connection states with the display layouts of display images. The display control unit 28 receives the connection state as an input recognized by the connection state recognition unit 26 and outputs a code indicating the display layout associated with the input connection state by using this table. The display control unit 28 then causes the display unit 22 to display a display image in the display layout corresponding to the output code.

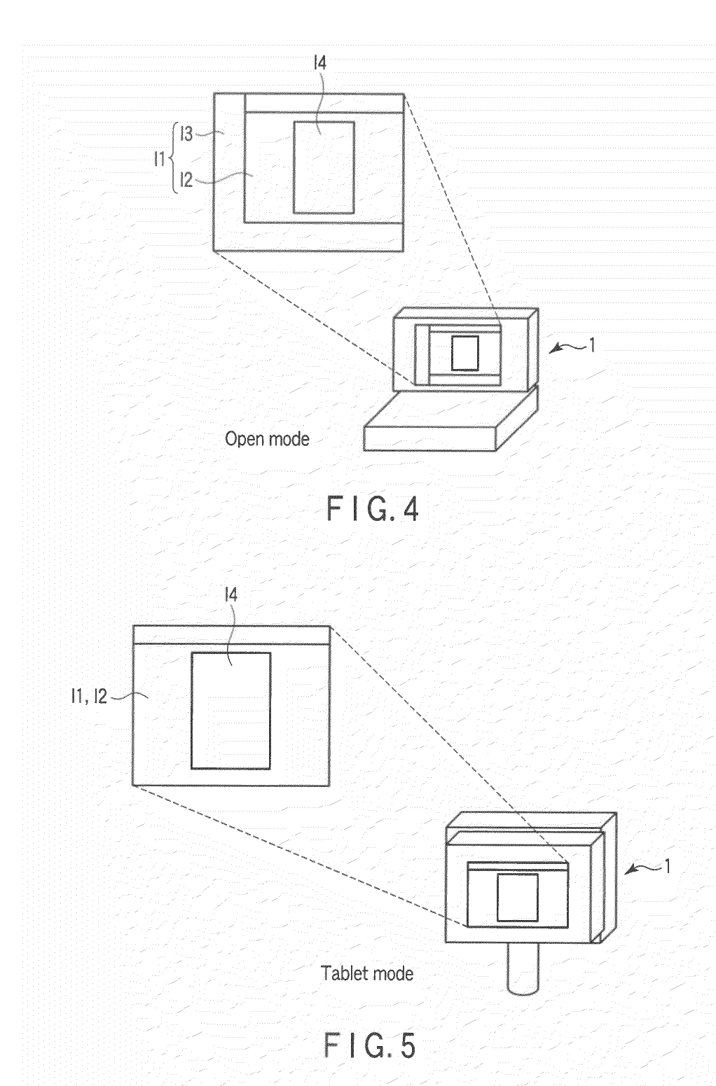

FIG. 4 is a view showing an example of the display layout of the display image I1 in the open mode. As shown in FIG. 4, in the display layout in the open mode, the image display area I2 and the panel display area I3 are set to have display positions, sizes, and shapes which are used for normal ultrasonic examination. A display layout in the open mode is set to allow to both display an ultrasonic image 14 and operate the ultrasonic diagnosis apparatus 1. To improve the visibility of the ultrasonic image 14, for example, the display position of the panel display area 13 is set at an end part of the screen 3, the size of the panel display area I3 is set to a size large enough to display switches necessary for the open mode, and the shape of the panel display area I3 is set to a portrait shape, a square shape, a landscape shape, or the like. It is possible to either set a display layout in advance or allow the operator to arbitrarily set a display layout.

FIG. 5 is a view showing an example of the display layout of the display image I1 in the tablet mode. In the display layout in the tablet mode, the display position, size, and shape of the image display area I2 are set so as to display only the image display area I2 on the entire screen 3. That is, the panel display area I3 does not exist.

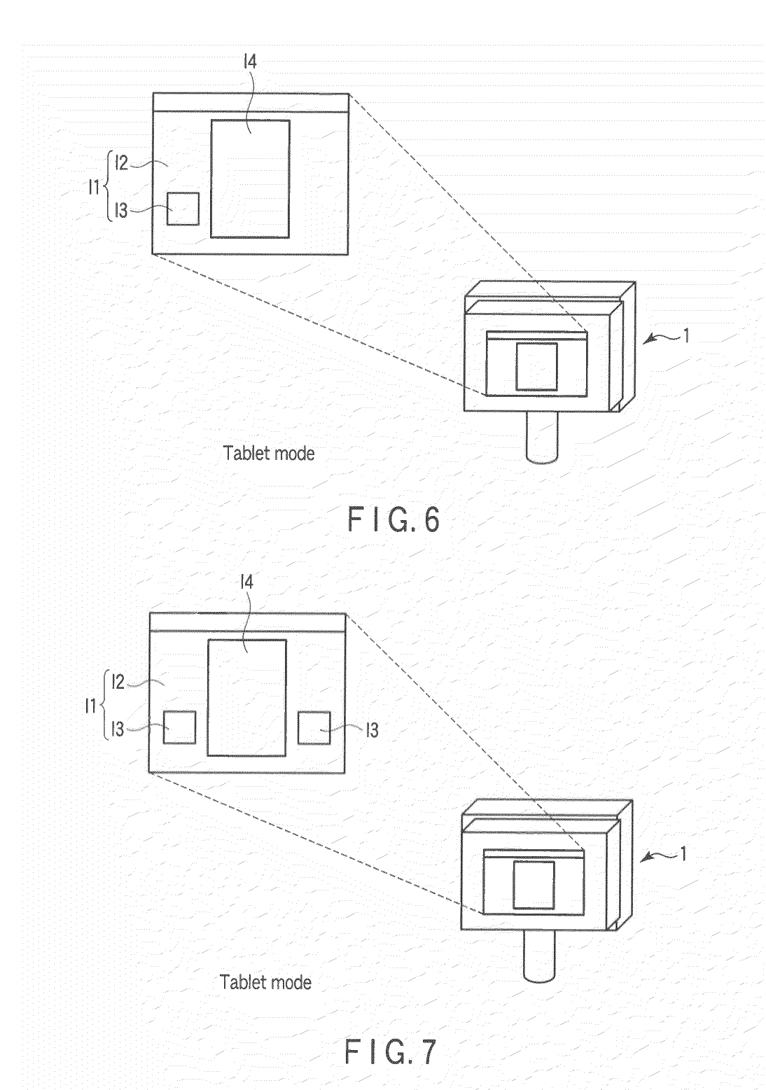

The tablet mode allows another display layout. FIG. 6 is a view showing another display layout in the tablet mode. As shown in FIG. 6, in another display layout in the tablet mode, the display position, size, and shape of the image display area I2 may be set to display the image display area I2 on the entire screen 3, and the display position, size, and shape of the panel display area I3 may be set to display the panel display area I3 at an end part of the screen 3. The display position of the panel display area I3 is set at an end part of the screen 3, the size of the panel display area I3 is set to a size large enough to display switches necessary for the tablet mode, and the shape of the panel display area I3 is set to a portrait shape, a square shape, a landscape shape, or the like. In this case, the number of switches displayed on the panel display area I3 is smaller than that in the open mode. It is possible to either set a display layout in advance or allow the operator to arbitrarily set a display layout.

FIG. 7 is a view showing still another display layout in the tablet mode. As shown in FIG. 7, in the display layout in this tablet mode, panel display areas 13 are displayed on two end parts of the screen 3.

Display layouts may be prepared for the panel display area in the tablet mode. For example, it is preferable to prepare a normal layout and a simplified layout as display layouts of the panel display area in accordance with the numbers of switches. The tablet mode is required to be simpler in operation than the open mode, and hence the number of switches on the normal layout in the tablet mode is smaller than that in the open mode. The simplified layout allows to set only the minimum necessary number of switches carefully selected in association with the operation of the ultrasonic diagnosis apparatus 1 itself. That is, the number of switches in the simplified layout is further smaller than that in the normal layout.

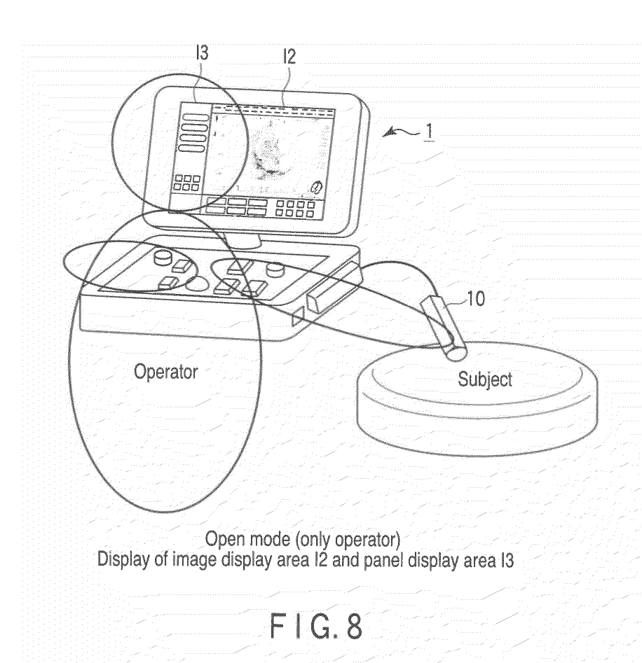

A clinical application example of the ultrasonic diagnosis apparatus according to the first example will be described next. In a clinical application, the ultrasonic diagnosis apparatus is mainly used in the open mode like that shown in FIG. 8. In this case, the operator keeps the ultrasonic diagnosis apparatus 1 on hand, and performs ultrasonic examination of a subject by using the ultrasonic probe 10 while observing the display image I1. At this time, the display image I1 includes the image display area I2 and the panel display area I3.

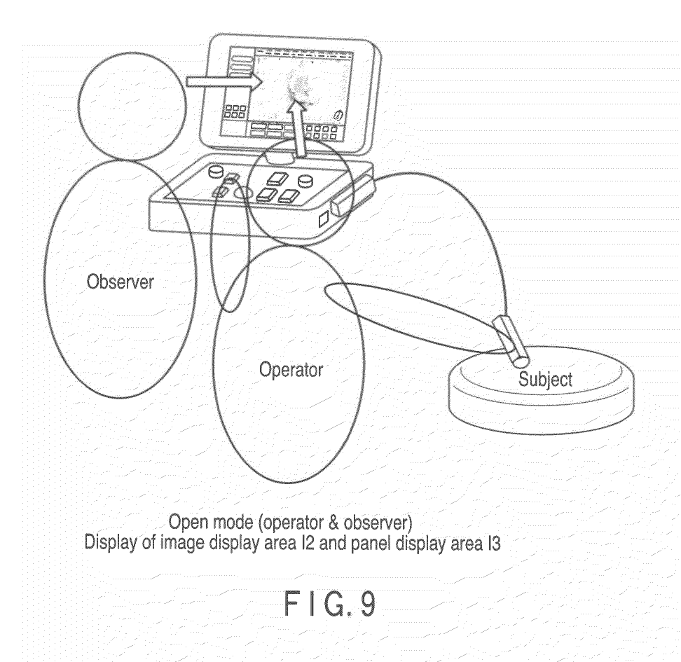

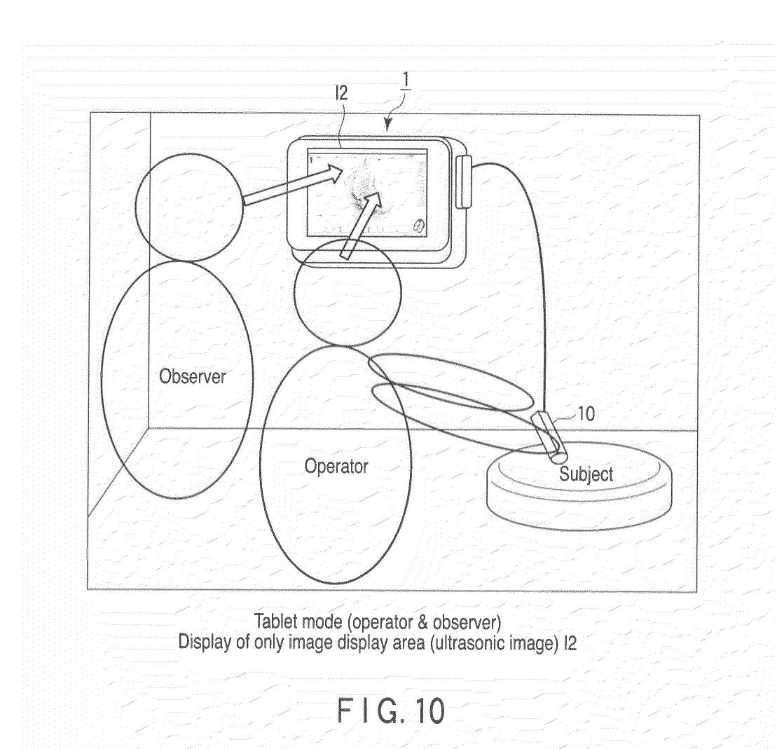

As shown in FIG. 9, a person other than the operator may perform examination while seeing the same display image I1. Persons other than the operator will be generically referred to as observers hereinafter. Such cases include, for example, a case in which a technician as an operator operates the ultrasonic probe 10, and a doctor as an observer checks an image. At this time, in a situation without any special equipment such as an external display, persons look in the small screen 3. This degrades the visibility of the display image I1 displayed on the screen 3.

In order to improve the visibility of the display image I1 for persons, the ultrasonic diagnosis apparatus 1 is placed at a position separated from the operator by a certain distance, as shown in FIG. 9. Methods of placing the ultrasonic diagnosis apparatus 1 include, for example, hooking the apparatus on a wall and placing the apparatus on a desk. In this case, the ultrasonic diagnosis apparatus 1 is used in the tablet mode. In other words, when the ultrasonic diagnosis apparatus 1 is used in the tablet mode, the apparatus is assumed to be placed at a position spaced away from the operator. When the ultrasonic diagnosis apparatus 1 is placed at a position spaced apart from the operator, it is assumed that the operator is concentrating on the operation of the ultrasonic probe 10 and on the screen 3 more than the operation of the ultrasonic diagnosis apparatus 1 itself. That is, the panel display area I3 on the display image is not required. For this reason, when the connection state recognition unit 26 recognizes that the apparatus is used in the tablet mode, the display control unit 28 erases the panel display area I3 on the display image I1, and displays the image display area I2 while enlarging the image display area I2 on the entire screen 3. Enlarging and displaying an ultrasonic image and additional data on the screen 3 in this manner will improve the visibility of the ultrasonic image. That is, when the operator changes the open mode to the tablet mode, the display control unit 28 enlarges the ultrasonic image to display it on the entire screen 3.

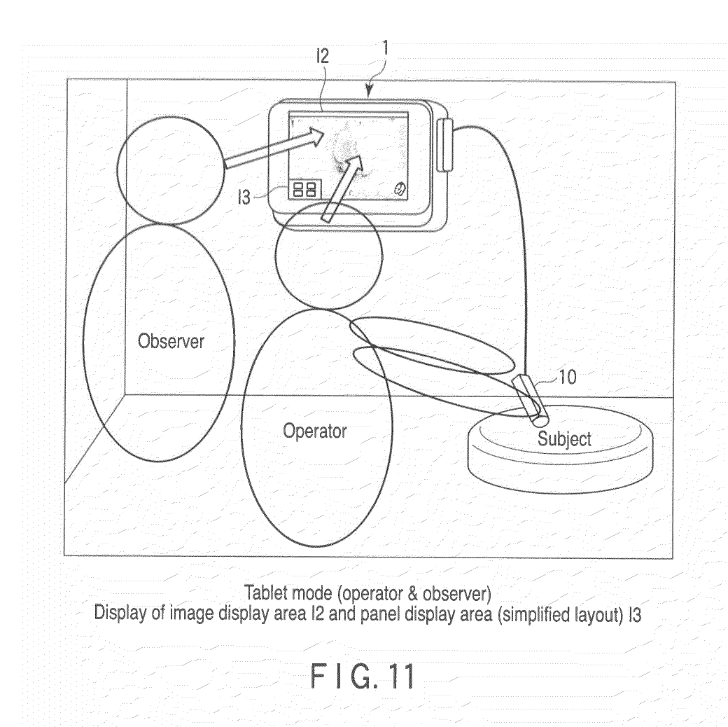

Even in the tablet mode, the operator may operate the ultrasonic diagnosis apparatus 1 itself. For this reason, even if the image display area I2 is enlarged and displayed, when the operation unit 24 detects that the screen is touched, the display control unit 28 may temporarily display the panel display area I3 on the screen 3, as shown in FIG. 11. In this case, in order to improve the visibility of the ultrasonic image, the display control unit 28 may display the panel display area I3 in the simplified layout upon reducing the panel display area I3 as compared with the normal display mode. As described above, when the panel display area I3 is to be reduced and displayed, it is preferable to carefully select the minimum necessary number of display switches associated with the operation of the ultrasonic diagnosis apparatus to ensure the visibility of the ultrasonic image.

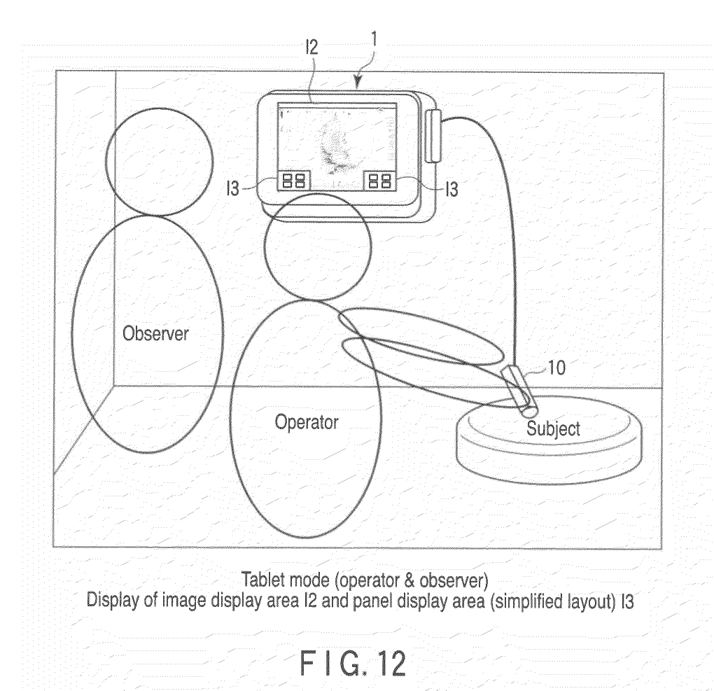

There is conceivable a case in which persons operate switches on the panel display area in the tablet mode. In this case, the display control unit 28 may display panel display areas 13 in parts on the screen 3. For example, as shown in FIG. 12, the display control unit 28 displays panel display areas 13 in a simplified layout at two end parts of the screen 3. Displaying the panel display areas 13 on the two end parts allows persons to easily operate on the panel display areas 13.

With the above arrangement, the ultrasonic diagnosis apparatus 1 changes at least one of the display positions, sizes, and shapes of the image display area and panel display area on the screen depending on whether the apparatus is used in the open mode or the tablet mode, i.e., whether the apparatus is located near the operator or spaced apart from the operator. This improves the visibility of an ultrasonic image and shortens the time associated with ultrasonic examination. In other words, the ultrasonic diagnosis apparatus 1 can display a display image in a display layout suitable for a connection state (use mode). The ultrasonic diagnosis apparatus 1 can display estimated parts of interest for different operators in an eye friendly manner in accordance with the use mode. Therefore, the ultrasonic diagnosis apparatus 1 according to the first example improves the throughput of ultrasonic examination. In addition, with an improvement in throughput, an improvement in the quality of ultrasonic examination can be expected.

### Second Example

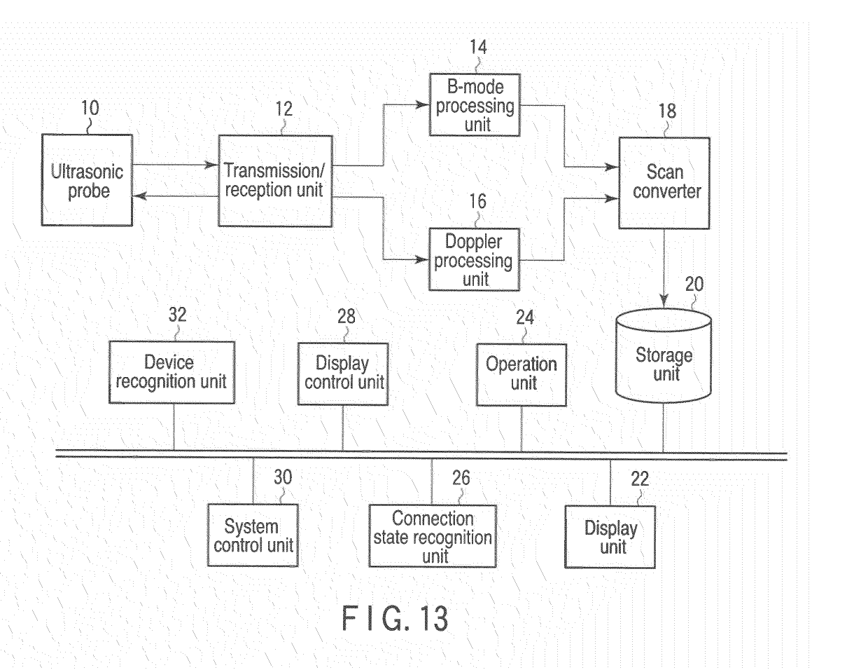

FIG. 13 is a functional block diagram of an ultrasonic diagnosis apparatus 1 according to the second example. As shown in FIG. 13, the ultrasonic diagnosis apparatus 1 according to the second example includes an ultrasonic probe 10, a transmission/reception unit 12, a B-mode processing unit 14, a Doppler processing unit 16, a scan converter 18, a storage unit 20, a display unit 22, an operation unit 24, a connection state recognition unit 26, a display control unit 28, a system control unit 30, and a device recognition unit 32. Note that the same reference numerals denote constituent elements having almost the same functions as those in the first example in the following description, and a repetitive description will be made only when required.

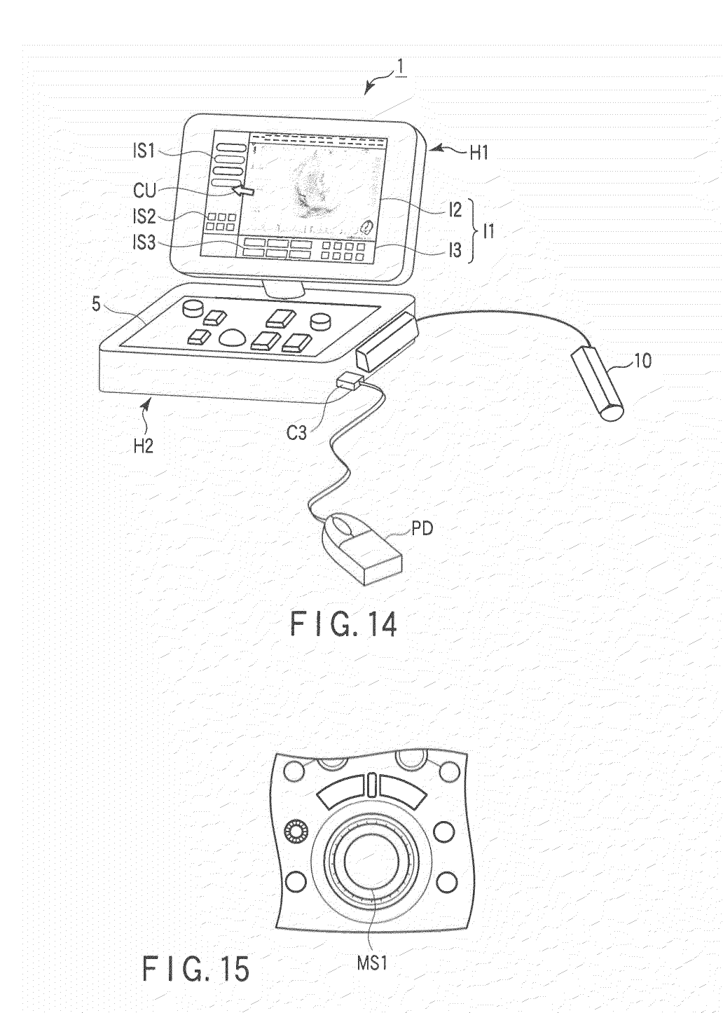

As shown in FIG. 14, the device recognition unit 32 recognizes whether an input device is connected to a device connection portion C3 provided for an operation housing H2. Upon recognizing that an input device is connected, the device recognition unit 32 recognizes the type of input device connected to the housing. For example, the device recognition unit 32 recognizes, as the type of input device, whether the connected input device is a pointing device such as a mouse or a keyboard. In addition, the device recognition unit 32 recognizes whether the connected mouse is wired or wireless.

When the device recognition unit 32 recognizes that a pointing device PD such as a mouse is connected, the display control unit 28 changes the display form of the screen. More specifically, when the pointing device PD is connected to the ultrasonic diagnosis apparatus 1, the display control unit 28 displays a cursor CU for GUI operation on the screen 3 or changes the size of characters or luminance. If the pointing device PD is connected, the display control unit 28 changes the display layout of a panel display area I3 from the normal layout or the simplified layout to an examination sequence layout. The examination sequence layout includes switches along an examination sequence. The display control unit 28 also changes the display form depending on whether the connected mouse is wired or wireless.

Display window switching processing according to the second example which is performed under the control of the system control unit 30 will be described in detail next.

As a pointing device in normal ultrasonic examination, a trackball MS1 like that shown in FIG. 15 or a touch panel is used. An OS such as Windows®, Mackintosh®, or Unix® generally uses a mouse for GUI operation. The operator can be expected to be skillful in using a mouse. That is, allowing to use a mouse as a pointing device makes it easier for the operator to operate the apparatus than allowing to use a trackball or touch panel as a pointing device.

When the device recognition unit 32 recognizes that the mouse PD is connected, the display control unit 28 displays the mouse cursor CU for GUI operation on the screen 3. When the device recognition unit 32 recognizes that the mouse PD is connected, the system control unit 30 accepts operation from the connected mouse PD. This will automatically recognize a mouse and display the mouse cursor CU on the general screen 3, thereby allowing the operator to perform all GUI operations with the mouse PD. In contrast, when the device recognition unit 32 recognizes that the mouse PD is disconnected from the ultrasonic diagnosis apparatus 1, the display control unit 28 erases the mouse cursor CU from the screen 3.

In other words, the device recognition unit 32 recognizes connection state between the operation housing H2 and pointing device. The device recognition unit 32 controls a display of cursor in accordance with the recognized connection state. The cursor is for operation of display panel by the pointing device.

In the tablet mode, the operation panel 5 hides itself in a display housing H1 to disable the operator to use the panel. That is, in the tablet mode, the operator must operate the ultrasonic diagnosis apparatus 1 with only switches IS displayed in the panel display area I3. It is therefore preferable to prepare a switch corresponding to each examination in an examination sequence and allow to execute processing for the examination assigned to the pressed switch. The display control unit 28 prepares an examination sequence layout for displaying switches along the examination sequence in the panel display area I3.

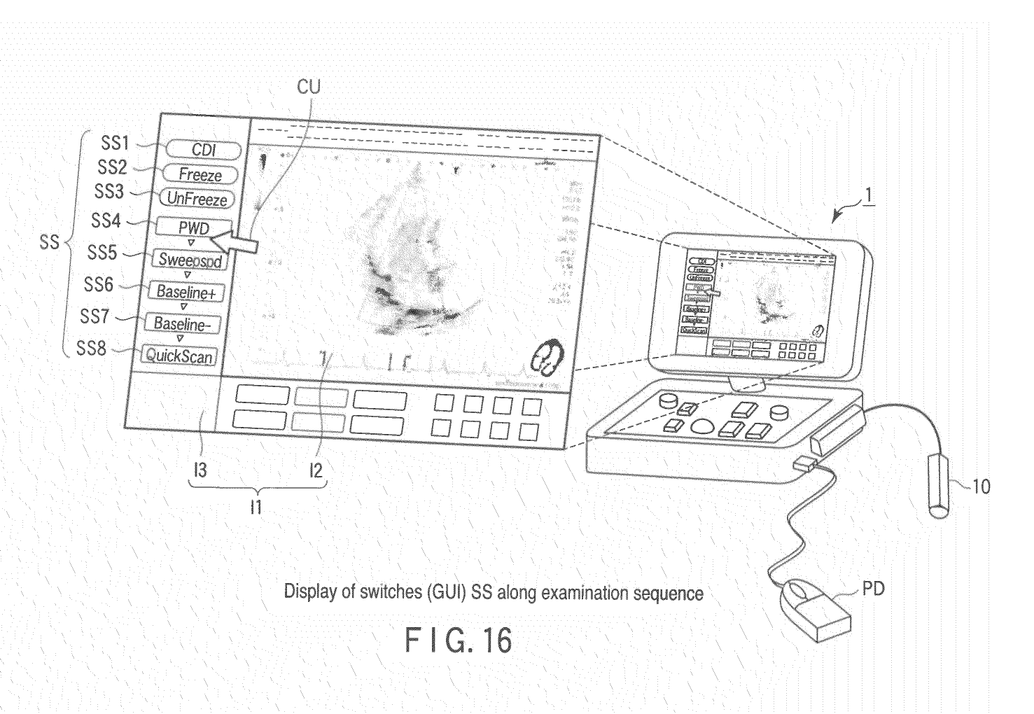

FIG. 16 is a view showing an example of an examination sequence layout. In an examination sequence layout, switches SS corresponding to the respective examinations in an examination sequence are prepared. The switches prepared include, in the order of examinations, a “CDI” switch SS1, “Freeze” switch SS2, “UnFreeze” switch SS3, “PWD” switch SS4, “Sweep spd” switch SS5, “Base line +” switch SS6, “Base line −” switch SS7, and “Quick scan” switch SS8. When the operator presses each of the switches SS1 to SS8, the system control unit 30 controls the respective units to execute the processing assigned to each of the switches SS1 to SS8.

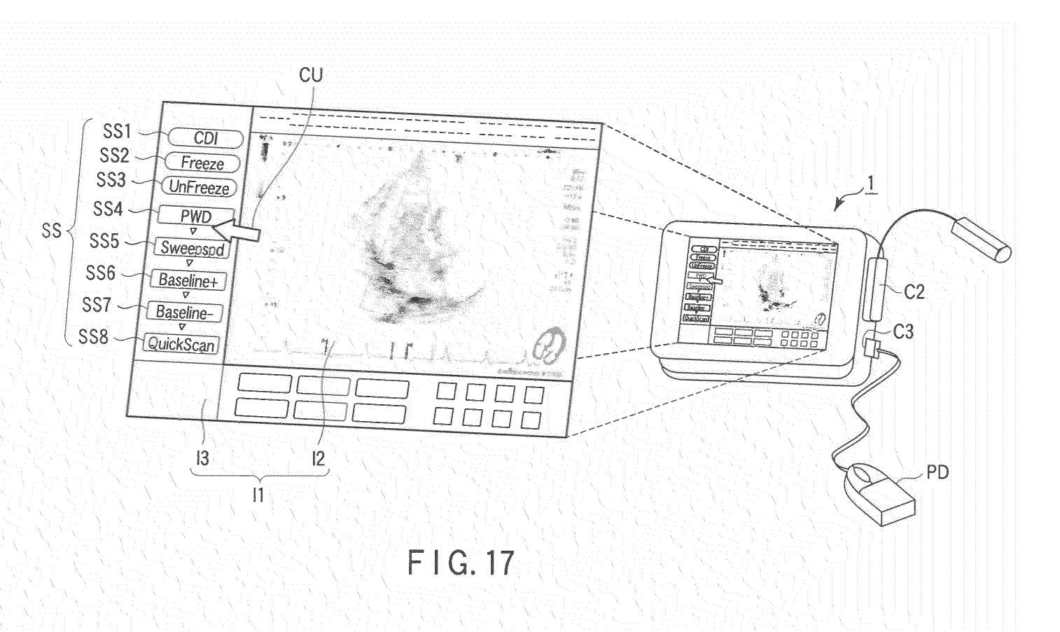

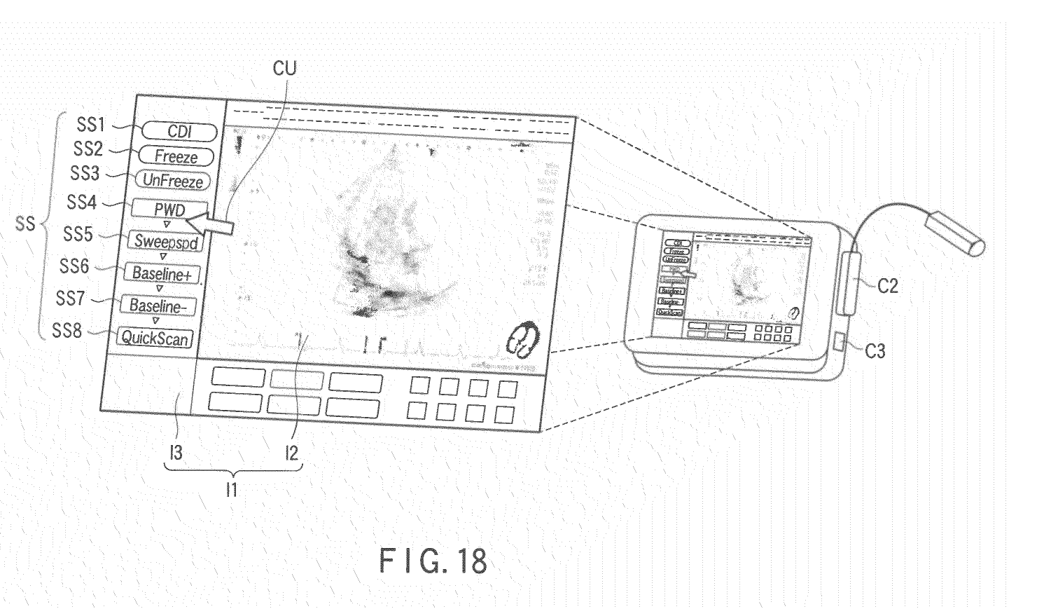

As shown in FIG. 17, the mouse PD may be connected to the apparatus in the tablet mode. In this case, it is preferable to provide a display layout which helps the progress of examination with simplified operation. When the tablet mode is set and the mouse PD is connected, therefore, the display control unit 28 may display the switches SS on the screen 3. As shown in FIG. 18, when the mouse PD is not connected in the tablet mode, the switches SS may be displayed.

In contrast, when the connection state recognition unit 26 recognizes that the tablet mode has changed to the open mode, the display control unit 28 changes the examination sequence layout to the normal mode. When the device recognition unit 32 recognizes that the mouse PD is disconnected in the tablet mode, the display control unit 28 changes the examination sequence layout to the normal layout or the simplified layout.

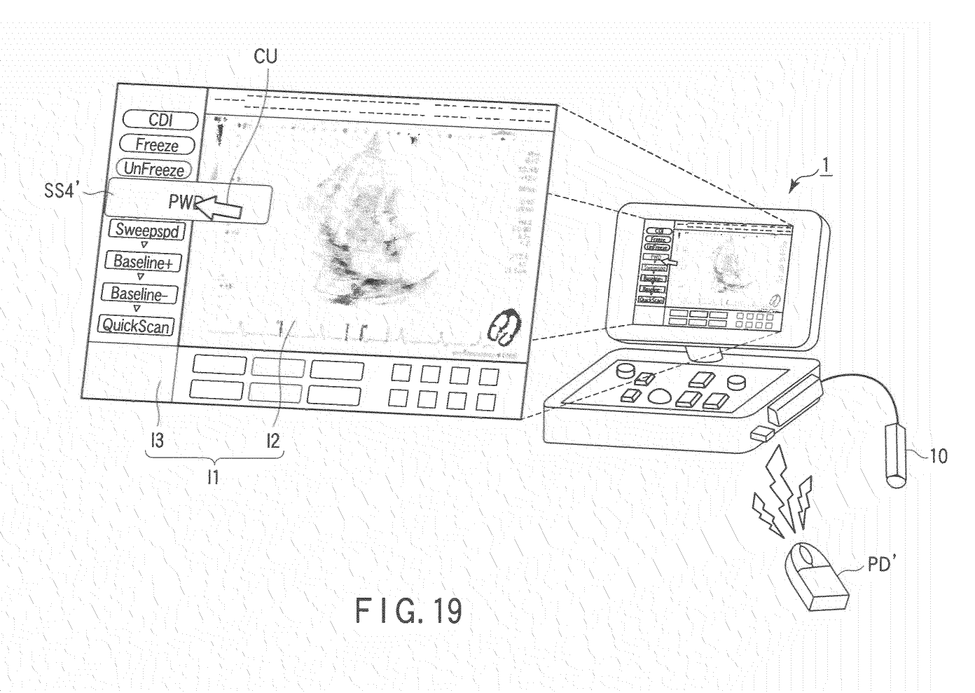

In addition, as shown in FIG. 19, it is assumed that when a wireless mouse PD′ is connected to the ultrasonic diagnosis apparatus 1, the operator is relatively far from the ultrasonic diagnosis apparatus 1. When the device recognition unit 32 recognizes that the wireless mouse PD′ is connected, the display control unit 28 changes the display form in the display image I1. When, for example, the operator selects a switch (e.g., SS4′) with the wireless mouse PD′, the display control unit 28 enlarges the selected switch and displays the enlarged switch. When the wireless mouse PD is connected, the display control unit 28 enlarges and displays characters in the display image I1 or the mouse cursor CU or increases the luminance distribution in the display image I1 to improve the visibility for the operator than when the wired mouse PD is connected.

With the above arrangement, the ultrasonic diagnosis apparatus 1 displays a mouse cursor on the screen or changes the display form on a display image to improve operability and visibility depending on whether a pointing device is connected. In addition, the ultrasonic diagnosis apparatus 1 switches layouts on the screen to improve operability depending on whether a pointing device is connected. Improving the operability of the ultrasonic diagnosis apparatus 1 in this manner shortens the time associated with ultrasonic examination. The notebook PC type ultrasonic diagnosis apparatus 1 according to the second example improves the throughput of ultrasonic examination. With an improvement in throughput, an improvement in the quality of examination can also be expected.

The above embodiments may apply to image display apparatus which has a notebook PC structure being able to change the open mode to the tablet mode and the tablet mode to the open mode. The image display apparatus according to this embodiment may be used for all existing sorts of ultrasonic examination, such as image processing for ultrasonic image, ultrasonic image interpretation, ultrasonic image observation, and report preparation, without ultrasonic scanning. The image display apparatus according to this embodiment has the similar structure of the ultrasonic diagnosis apparatus shown in FIG. 1, FIG. 2, and FIG. 14. The image display apparatus according to this embodiment includes at least function of the storage unit 20, the display unit 22, the operation unit 24, the connection state recognition unit 26, the display control unit 28, the system control unit 30, and the device recognition unit 32.

Therefore, the ultrasonic diagnosis apparatus having a notebook PC structure, the image display apparatus, the image display method, and the display method improves the throughput of ultrasonic examination.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An ultrasonic diagnosis apparatus comprising:
an ultrasonic probe configured to transmit and receive an ultrasonic wave;
a generating unit configured to generate ultrasonic image data based on an echo signal from the ultrasonic probe;
a first housing connected to the ultrasonic probe and including an operation panel for issuing an operation instruction associated with ultrasonic examination;
a second housing connected to the first housing and including a screen;
a display unit configured to display a display image on the screen, the display image including at least one of an image display area for the generated ultrasonic image and a panel display area for a display panel;
a recognition unit configured to recognize a connection state between the first housing and the second housing; and
a display control unit configured to change at least one of display positions, sizes, and shapes of the image display area and the panel display area on the display image in accordance with the recognized connection state.

2. The apparatus according to claim 1, wherein the recognition unit recognizes whether the connection state is a first connection state or a second connection state, the first connection state being in which the first housing and the second housing are supported so as to make the operation panel face the screen, the second connection state being in which the first housing and the second housing are folded so as not to make the operation panel face the screen.

3. The apparatus according to claim 2, wherein when the recognition unit recognizes the second connection state, the display control unit enlarges the ultrasonic image as compared when the recognition unit recognizes the first connection state.

4. The apparatus according to claim 2, wherein when the recognition unit recognizes the second connection state, the display control unit reduces the panel display area as compared when the recognition unit recognizes the first connection state.

5. The apparatus according to claim 2, wherein when the recognition unit recognizes the second connection state, the display control unit decreases the number of switches in the panel display area as compared when the recognition unit recognizes the first connection state.

6. The apparatus according to claim 2, wherein when the recognition unit recognizes the second connection state, the display control unit displays the panel display areas on both sides of the screen.

7. An ultrasonic diagnosis apparatus comprising:
an ultrasonic probe configured to transmit and receive an ultrasonic wave;
a generating unit configured to generate ultrasonic image data based on an echo signal from the ultrasonic probe;
a first housing connected to the ultrasonic probe and comprising a connection portion for a pointing device to issue an operation instruction associated with ultrasonic examination;
a second housing connected to the first housing and including a screen;
a display unit configured to display an image display area and a panel display area on the screen, the image display area being for the generated ultrasonic image, the panel display area being for a display panel;
a recognition unit configured to recognize whether the pointing device is connected to the connection portion; and
a display control unit configured to display a cursor on the screen when the recognition unit recognizes that the pointing device is connected, the cursor being for operation of the display panel by the pointing device.

8. The apparatus according to claim 7, wherein the display control unit switches layouts in the panel display area depending on whether the pointing device is wired or wireless.

9. The apparatus according to claim 8, wherein the display control unit display a switch on the panel display area when the pointing device is wireless, the switch being corresponding to each examination in an examination sequence.

10. The apparatus according to claim 8, wherein the display control unit enlarges a selected switch on the panel display area when the pointing device is wireless.

11. The apparatus according to claim 8, wherein the display control unit enlarges a mouse cursor when the pointing device is wireless.

12. An image display apparatus comprising:
a storage unit configured to storage ultrasonic image data generated by an ultrasonic diagnosis apparatus;
a first housing including an operation panel for issuing an operation instruction associated with ultrasonic examination;
a second housing connected to the first housing and including a screen;
a display unit configured to display a display image on the screen, the display image including at least one of an image display area for the generated ultrasonic image and a panel display area for a display panel;
a recognition unit configured to recognize a connection state between the first housing and the second housing; and
a display control unit configured to change at least one of display positions, sizes, and shapes of the image display area and the panel display area on the display image in accordance with the recognized connection state.

13. An image display apparatus comprising:
a storage unit configured to storage ultrasonic image data generated by an ultrasonic diagnosis apparatus;
a first housing including a connection portion for a pointing device to issue an operation instruction associated with ultrasonic examination;
a second housing connected to the first housing and including a screen;
a display unit configured to display an image display area and a panel display area on the screen, the image display area being for the generated ultrasonic image, the panel display area being for a display panel;
a recognition unit configured to recognize whether the pointing device is connected to the connection portion; and
a display control unit configured to display a cursor on the screen when the recognition unit recognizes that the pointing device is connected, the cursor being for operation of the display panel by the pointing device.

14. An image display method of ultrasonic diagnosis apparatus including; an ultrasonic probe configured to transmit and receive an ultrasonic wave; a generating unit configured to generate ultrasonic image data based on an echo signal from the ultrasonic probe; a first housing connected to the ultrasonic probe and including an operation panel for issuing an operation instruction associated with ultrasonic examination; a second housing connected to the first housing and including a screen; and a display unit configured to display a display image on the screen, the display image including at least one of an image display area for the generated ultrasonic image and a panel display area for a display panel, An image display method comprising:
recognizing a connection state between the first housing and the second housing; and
changing at least one of display positions, sizes, and shapes of the image display area and the panel display area on the display image in accordance with the recognized connection state.

15. An image display method of ultrasonic diagnosis apparatus including; an ultrasonic probe configured to transmit and receive an ultrasonic wave; a generating unit configured to generate ultrasonic image data based on an echo signal from the ultrasonic probe; a first housing connected to the ultrasonic probe and including a connection portion for a pointing device to issue an operation instruction associated with ultrasonic examination; a second housing connected to the first housing and including a screen; and a display unit configured to display an ultrasonic image display area and a panel display area on the screen, the image display method comprising:
recognizing whether the pointing device is connected to the connection portion; and
displaying a cursor on the screen when the recognition unit recognizes that the pointing device is connected, the cursor being for operation of the display panel by the pointing device.

16. A display method comprising:
recognizing a connection state between a first housing and a second housing, the first housing including an operation panel for issuing an operation instruction associated with ultrasonic examination, the second housing including a screen; and
changing at least one of display positions, sizes, and shapes of an ultrasonic image display area and a panel display area on the display image in accordance with the recognized connection state.

17. A display method comprising:
recognizing a connection state between a pointing device and housing, the pointing device being for issuing an operation instruction associated with ultrasonic examination, the housing being for issuing an operation instruction associated with ultrasonic examination; and
controlling a display of a cursor in accordance with the recognized connection state, the cursor being for operation of display panel by the pointing device.
